(19) United States
(12) Patent Application Publication
(10) Pub. No.: US 2017/0095643 A1
(43) Pub. Date: Apr. 6, 2017

(54) SYSTEMS, DEVICES, KITS, AND METHODS FOR DELIVERING THERAPEUTIC AGENTS

(71) Applicant: BOSTON SCIENTIFIC SCIMED, INC., MAPLE GROVE, MN (US)

(72) Inventor: AIDEN FLANAGAN, CO. GALWAY (IE)

(73) Assignee: BOSTON SCIENTIFIC SCIMED, INC., MAPLE GROVE, MN (US)

(21) Appl. No.: 15/210,585

(22) Filed: Jul. 14, 2016

Related U.S. Application Data
(60) Provisional application No. 62/235,975, filed on Oct. 1, 2015.

(51) Int. Cl. A61M 25/00; A61M 5/19

(52) U.S. Cl. CPC A61M 25/0084; A61M 25/0071; A61M 5/19; A61M 2210/125; A61M 2207/00; A61M 2025/0073; A61M 2025/0091; B29C 53/583

(57) ABSTRACT

An injection catheter includes a drive mechanism, an elongate, flexible tubular body, and a first inner member. The tubular body has a distal end portion, a proximal end portion, and defines a first lumen extending therebetween. The first inner member includes a proximal end coupled to the drive mechanism and is rotatably disposed within the first lumen. The first inner member and the first lumen define an annular gap therebetween configured to deliver a therapeutic agent contained therein from the proximal end portion to the distal end portion of the tubular body when the first inner member is rotated within the lumen such that shear stresses exerted on the therapeutic agent are less than 500 pascals.

### CROSS-REFERENCE TO RELATED APPLICATIONS

This application claims priority under 35 U.S.C. §119(e) to U.S. Provisional Application No. 62/235,975, filed Oct. 1, 2015, the entire disclosure of which is herein incorporated by reference.

### TECHNICAL FIELD

This document relates to systems, devices, kits, and methods relating to delivering a therapeutic agent (e.g., a therapeutic gel) to a target area in a patient's body.

### BACKGROUND

Heart failure due to damaged cardiac tissue is a significant health care issue. It has been proposed to treat the damaged tissue directly with a therapeutic agent designed to help regenerate the damaged tissue. An example of a therapeutic agent proposed for this use is stem cells. The stem cells would be delivered in the form of a gel to the site of the damaged tissue. The gels, however, can have relatively high viscosities. Therefore, administering the gel through a conventional syringe would subject the stem cells to relatively high pressure, potentially damaging the cells and compromising their therapeutic efficacy.

### SUMMARY

Disclosed herein are various embodiments of systems, devices, kits, and methods related thereto for delivering a therapeutic agent, such as a therapeutic gel, into a patient's body. In particular, some embodiments provided herein include devices, such as injection catheters, for delivering the therapeutic agent into tissue (e.g., cardiac tissue).

In Example 1, an injection catheter includes a drive mechanism, an elongate, flexible tubular body, and a first inner member. The tubular body has a distal end portion, a proximal end portion, and a first lumen extending therebetween. The first inner member includes a proximal end coupled to the drive mechanism and rotatably disposed within the first lumen. The injection catheter is characterized by the first inner member and the first lumen defining an annular gap therebetween configured to deliver a therapeutic agent contained therein from the proximal end portion to the distal end portion of the tubular body when the first inner member is rotated within the lumen such that shear stresses exerted on the therapeutic agent are less than 500 pascals.

In Example 2, the injection catheter of Example 1, wherein first inner member includes a helical feature formed along at least a portion of the first inner member and the annular gap is partially defined by an interstitial channel of the helical feature.

In Example 3, the injection catheter of Example 1 or Example 2, wherein the injection catheter further includes a needle coupled to the distal end of the tubular body and a manifold coupled to the proximal end of the tubular body, wherein the manifold is connectable to multiple therapeutic agent supply sources.

In Example 4, the injection catheter of any one of Examples 1-3, wherein the tubular body further defines a second lumen extending from the proximal end portion to the distal end portion and a second inner member that has a proximal end coupled to the drive mechanism, wherein the second inner member is rotatably disposed within the second lumen.

In Example 5, the injection catheter of any one of Examples 1-4, further including a distal portion between the needle and the distal end of the tubular body, the distal portion including an inner junction that joins the first and second lumens of the tubular body together into a distal lumen.

In Example 6, the injection catheter of Example 5, wherein the distal lumen is located distal to the inner junction and defines a curvilinear fluid pathway adapted for mixing at least two therapeutic agents within the distal lumen prior to dispensing the mixed agents from the needle.

In Example 7, the injection catheter of any one of Examples 1-6, wherein the annular gap is adapted to receive a therapeutic agent having a viscosity ranging from about 100 centipoises to about 100,000 centipoises.

In Example 8, the injection catheter of any one of Examples 1-7, wherein the helical feature of the first inner member is formed by threads having an amplitude ranging from about 0.1 millimeters to about 0.9 millimeters, a pitch ranging from about 0.1 millimeters to about 2.0 millimeters, and a thread groove width ranging from about to about 0.1 millimeters to about 3 millimeters.

In Example 9, the injection catheter of any one of Examples 1-8, wherein the needle has a needle size ranging from about 28 gauge to about 15 gauge.

In Example 10, the injection catheter of any one of Examples 1-9, wherein the inner member includes nitinol, stainless steel, stainless steel alloys, cobalt-based alloys, titanium, titanium alloys, or combinations thereof.

In Example 11, an injection catheter system including the injection catheter of any of Examples 1-10, the system further including two sensors and a feedback controller, wherein each sensor is coupled to a therapeutic agent supply source to monitor volume of a therapeutic agent within the supply source.

In Example 12, a kit including the injection catheter of any of Examples 1-10, the kit further including at least two therapeutic agent components that are intended to be mixed equally at the site of injection to crosslink to form a therapeutic gel.

In Example 13, an injection catheter includes an elongate, flexible tubular body having a distal end portion, a proximal end portion, and at least one lumen extending therebetween and a means for delivering a therapeutic agent within the at least one lumen from the proximal end portion to the distal end portion of the tubular body such that shear stresses exerted on the therapeutic agent are less than 500 pascals (Pa).

In Example 14, a method of manufacturing an injection catheter, the method characterized by a deformable winding of a first elongate member about a second elongate member such that the first elongate member forms a helical feature about the second elongate member.

In Example 15, the method of Example 14, wherein the first and second elongate members are bonded together using at least one of adhesive bonding, soldering, and welding.

In Example 16, the method of Example 14 or 15, wherein the first elongate member is wound about the second elongate member such that the first elongate member applies a compressional force on the second member such that an interference fit is created between the first and second elongate members.

In Example 17, the method of any one of Examples 14-16, wherein the first elongate member is wound about the second elongate member with a variable pitch.

In Example 18, the method of any one of Examples 14-17, wherein the first elongate member has a round, a square, an oval, or a rectangular cross-sectional shape.

In Example 19, the method of any one of Examples 14-18, further including a laser cutting process to shape the first elongate member into a final form.

In Example 20, the method of any one of Examples 14-19, further including obtaining a third elongate member and deformably winding the third elongate member about the second elongate member.

In Example 21, an injection catheter includes a drive mechanism, an elongate tubular body, a needle, and a manifold. The tubular body can include a distal end, a proximal end, and at least two lumens defined by luminal walls of the body. The needle can be coupled to the distal end of the elongate tubular body. The manifold can be coupled to the proximal end of the tubular body, and connectable to multiple therapeutic agent supply sources. The injection catheter may include first and second inner members each rotatably disposed in one of the two lumens. Each inner member can include a helical feature formed along at least a portion of the inner member. The luminal walls of the tubular body and the helical feature of each inner member can define an interstitial cavity therebetween. The proximal end of each inner member can be coupled to the drive mechanism such that the first and the second inner members are each rotatable within a corresponding lumen of the tubular body. The injection catheter may be characterized by the interstitial cavities forming first and second fluid pathways for delivering a therapeutic agent from the manifold to the needle.

In Example 22, the injection catheter of Example 21, wherein the injection catheter is adapted for delivering the therapeutic agent by rotation of the inner member within the tubular body such that shear stresses exerted on the therapeutic agent are less than 500 pascals (Pa).

In Example 23, the injection catheter of Example 21 or 22, wherein the injection catheter is adapted for delivering a first therapeutic agent within a first continuous pathway defined by the interstitial cavity of a first lumen and the first inner member and a second therapeutic agent in a second continuous pathway defined by the interstitial cavity of a second lumen and the second inner member.

In Example 24, the injection catheter of any one of Examples 21-23, further including a distal portion between the needle and the distal end of the elongate tubular body, the distal portion including an inner junction that joins the two lumens of the tubular body together into a distal lumen.

In Example 25, the injection catheter of Example 24, wherein the distal lumen is located distal to the inner junction and defines a curvilinear fluid pathway adapted for mixing at least two therapeutic agents within the distal lumen prior to dispensing the mixed agents from the needle.

In Example 26, the injection catheter of any one of Examples 21-25, wherein a fluid pathway is formed by each interstitial cavity, wherein each fluid pathway is defined a minimum diameter of each interstitial cavity and the lumen walls of the tubular body.

In Example 27, the injection catheter of any one of Examples 21-26, wherein each interstitial cavity is adapted to receive a therapeutic agent having a viscosity ranging from about 100 centipoises (cP) to about 100,000 cP.

In Example 28, the injection catheter of any one of Examples 21-27, wherein the helical feature of each inner member is formed by threads having an amplitude ranging from about 0.1 millimeters to about 0.9 millimeters, a pitch ranging from about 0.1 millimeters to about 2.0 millimeters, and a thread groove width ranging from about to about 0.1 millimeters to about 3 millimeters.

In Example 29, the injection catheter of any one of Examples 21-28, wherein the helical feature of each inner member forms an auger.

In Example 30, the injection catheter of any one of Examples 21-29, wherein the needle has a needle size ranging from about 28 gauge to about 15 gauge.

In Example 31, the injection catheter of any one of Examples 21-30, wherein the inner member includes nitinol, stainless steel, stainless steel alloys, cobalt-based alloys, titanium, titanium alloys, or combinations thereof.

In Example 32, an injection catheter system includes the injection catheter of any of Examples 21-31. The system further includes two sensors and a feedback controller, wherein each sensor can be coupled to a therapeutic agent supply source to monitor volume of a therapeutic agent within the supply source.

In Example 33, an injection catheter includes a drive mechanism, an elongate tubular body, a needle, and a manifold. The tubular body has a distal end, a proximal end, and a lumen defined by luminal walls of the body, in which at least a portion of the lumen defines an elongate lumen. The needle can be coupled to the distal end of the elongate tubular body. The manifold can be coupled to the proximal end of the tubular body, and connectable to a therapeutic agent supply source. The inner member can be rotatably disposed within the lumen. At least a portion of the inner member can include a helically-shaped portion disposed within the elongate lumen. The luminal walls of the tubular body and the helically-shaped portion of the inner member can define a plurality of discrete annular gaps therebetween. The proximal end of each inner member can be coupled to the drive mechanism such that the inner member is rotatable within the lumen of the tubular body. The injection catheter can be characterized by the plurality of discrete annular gaps forming a fluid pathway for delivering a therapeutic agent from the manifold to the needle.

In Example 34, a method of manufacturing an injection catheter includes deformably winding a first elongate member about a second elongate member such that the first elongate member forms a helical feature around the second elongate member;

In Example 35, the method of Example 34, wherein the first and second elongate members are bonded together using at least one of adhesive bonding, soldering, and welding.

The embodiments of the devices, systems and methods provided herein may provide one or more of the following advantages. First, some embodiments of the systems and devices provided herein can be configured for delivering therapeutic agents containing an active therapeutic agent (e.g., stem cells) without compromising the therapeutic efficacy of the therapeutic agent, or minimizing potential damage to the therapeutic agent caused by exposing the therapeutic agents to shear stresses when the agents pass through a lumen during a therapeutic injection. More specifically, in some cases, the embodiments of the devices provided herein can deliver therapeutic agents by limiting the amount of the shear stresses being exerted on the active therapeutic agent. Certain types of therapeutics agents, such as stem cells, are highly sensitive to shear stress and can become damaged when subjected to a threshold level of shear stress. In some cases, devices provided herein can include a catheter having an elongate, tubular shaft defining one or more lumens configured for receiving an inner member that includes a helical feature (which will be discussed in greater detail with FIG. 2D) for delivering (e.g., pumping) a viscous therapeutic agent from a proximal end to a distal end of the catheter device or system. In some cases, a catheter containing the inner member can be adapted for transporting a gel or gel-like substance within one or more interstitial cavities of the inner member, each interstitial cavity partially forming the annular gap. The inner member can be rotated to advance the agent in an axial direction rather than using a high injection pressure to push the therapeutic agent through the catheter lumen. In some cases, the catheter can deliver the therapeutic agent from the proximal portion (e.g., the manifold) to a distal tip (e.g., the needle) of the device without exposing the therapeutics to shear stresses greater than 500 pascals (Pa) during device use. Accordingly, the systems and devices provided herein may be used to deliver therapeutics contained in highly viscous fluids, e.g., fluids having a viscosity of about 2 pascal-seconds (or 2000 cP), using a percutaneous, transluminal delivery catheter and methods related to.

In contrast, an injection catheter device that includes a delivery lumen, but lacks the inner member described herein, may require a substantially higher pressure to deliver viscous fluids. For example, an injection catheter having a working length of approximately 150 centimeters and a lumen diameter of at least 1 millimeter may need an injection pressure of about 1 megapascal (Mpa) for delivering a viscous fluid of about 2 pascal-seconds (2000 cP) at a reasonable flow rate, such as a flow rate of about 0.5 milliliters per minute. Under such pressure constraints, a practitioner would likely need to apply an unrealistic force of about 350 N (79 pounds-force) when using a hand-operated syringe with a 10-millimeter inner diameter. As a result, the practitioner would need to use a machine to apply such high pressures, which could damage the therapeutic agent, or, alternatively, use a catheter design that includes a larger lumen diameter, which would likely impact the flexibility characteristics of the catheter.

Second, certain methods provided herein can allow for rapid and versatile manufacturing of a device or a system described herein. For example, in some cases, methods provided herein can include a winding process and an optional bonding process for quickly forming key components, such as a rotatable inner component (e.g., inner member). Methods provided herein may be easily adjusted to accommodate the manufacturing of alternative designs. For example, the methods provided herein may be easily adjusted to change design parameters of the inner member, for example, the pitch and amplitude of the helical feature.

Third, some of the embodiments provided herein include an inner member that includes a helical feature configured for providing precise control of an injection during device use. For instance, a practitioner may desire to immediately change the injection flow rate (e.g., immediately start, stop or change the flow rate) of an injection with a minimal lag time between actuating the flow rate change at a proximal end of the device and observing the flow rate change at a distal end of the device. In effect, embodiments provided herein that include the inner member may provide a practitioner with precise control of important injection parameters, such as an injection time and an injection volume of the therapeutic agent, because the injection parameters are largely dependent on torque forces applied at the proximal end of the device that can provide, in some cases, a one-to-one movement of the inner component between its proximal and distal ends. Precise control of the injection parameters can allow the practitioner to dispense a desired volume of a therapeutic agent into tissue as well as help prevent undesirable risks, such as embolic risk, associated with an injectant dispensing from a needle after the needle has been withdrawn from tissue during a medical procedure.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 illustrates an exemplary embodiment of an injection catheter system provided herein for delivering a therapeutic agent to a treatment location.

FIGS. 2A-2D provide additional illustrations the injection catheter system of FIG. 1 that show features in greater detail. FIG. 2A is a perspective view of the injection catheter of FIG. 1. FIG. 2B is a magnified, cross-sectional side view of a distal portion of the injection catheter system. FIG. 2C is a transverse cross-section of the injection catheter system. FIG. 2D is a magnified cross-sectional view of a proximal portion of the injection catheter system.

FIGS. 3-6 are transverse cross-sectional views and side views of various examples of an inner member of an injection catheter device provided herein.

FIG. 7 is a magnified, cross-sectional side view at a distal portion of an alternative embodiment of an injection catheter device provided herein.

FIG. 8 provides a magnified, cross-sectional side view at a distal portion of an alternative embodiment of an injection catheter device provided herein.

FIGS. 9A-9D are illustrations of an alternative embodiment of an injection catheter device provided herein. FIG. 9A is a perspective view of the device that shows an inner member disposed within a lumen of a tubular body of the device. FIG. 9B provides a series of side and cross-sectional views to illustrate how the inner member translates within the lumen of the tubular body when rotated therein. FIG. 9C is a perspective view of the inner member with discrete volumes of therapeutic agent that would be disposed within gaps formed between the inner member and the lumen of the tubular body. FIG. 9D is a perspective view showing only the discrete volumes of therapeutic agent being delivered by the injection catheter device.

FIG. 10 is an illustration of how to make an inner member of an injection catheter device provided herein.

Like reference symbols in the various drawings indicate like elements.

### DETAILED DESCRIPTION

In some cases, therapeutic agents and liquids, delivered using methods, devices, systems, and kits provided herein, can have a relatively high viscosity, which can complicate the injection of these compositions. Certain therapeutic agents can include shear-sensitive therapeutic agents, such as cells, which are vulnerable to high pressures that create high shear stresses and cause damage to the shear-sensitive materials. Delivering shear-sensitive materials (e.g., therapeutic agents) through a long catheter lumen, however, often requires high pressures that can damage the agents, reducing the therapeutic agent effectiveness, or even rendering the therapeutic agent inactive, when it is injected from an injection catheter system. Methods, devices, systems, and kits provided herein include a novel catheter design for delivering therapeutic agents from a proximal end to at a distal end (e.g., a needle) of the injection device or system in a manner that reduces or eliminates shear stresses subjected on the therapeutic agents. In some cases, the methods, systems, and devices provided herein can be adapted to generate shear less than 500 Pa on the therapeutic composition when delivering the therapeutic agent. A threshold stress value for damaging certain types of therapeutic agents, e.g., red blood cells, is approximately 500 Pa. See Yen J H et al., The Effect Of Turbulent Viscous Shear Stress On Red Blood Cell Hemolysis, J. Artif. Organs, 17:2, 178-85 (2014); R. P. Bacher et al., Hemolysis in Capillary Flow, J. Lab. Clin. Med., 76, 485-496 (1970); and Rooney J. A., Hemolysis Near an Ultrasonically Pulsating Gas Bubble, Science, 169, 869-871 (1970).

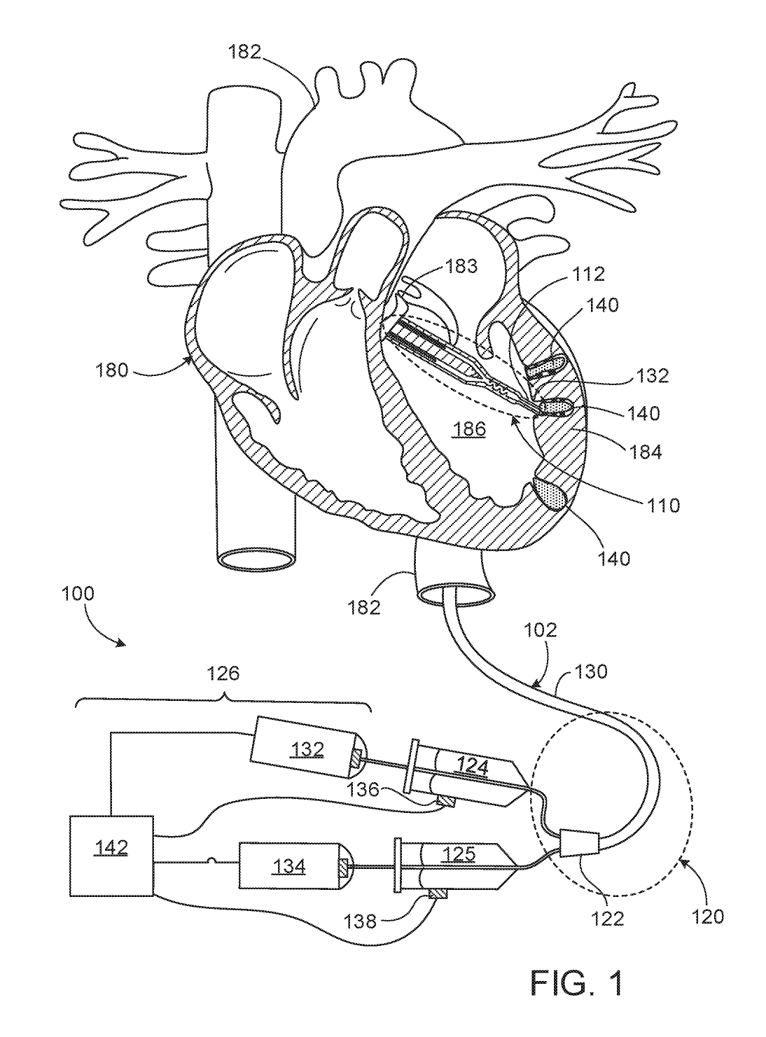

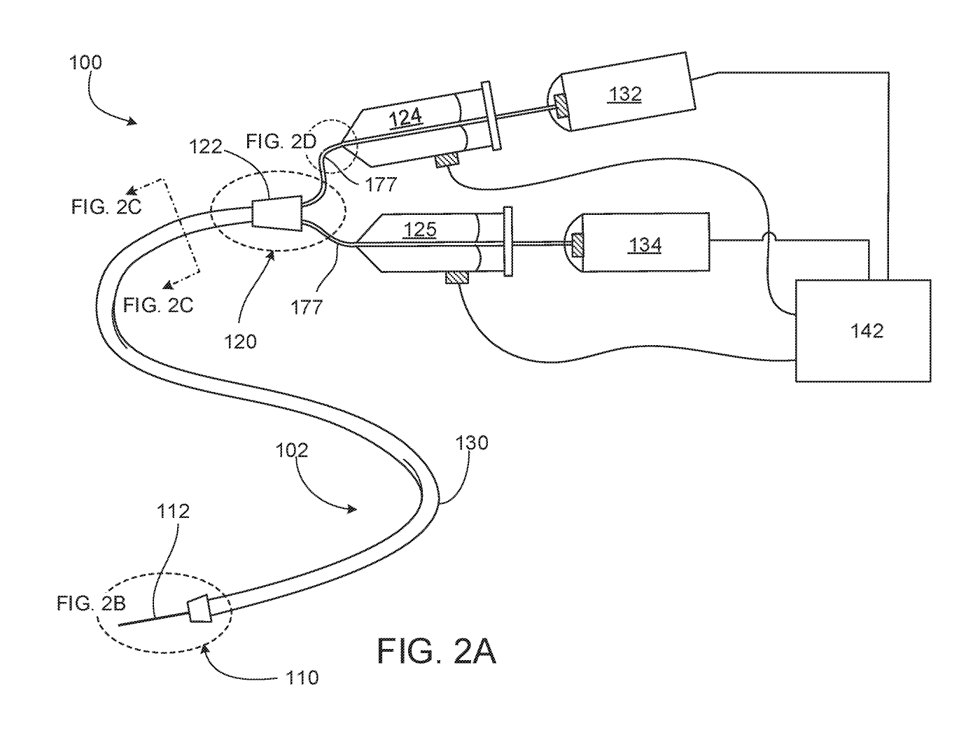

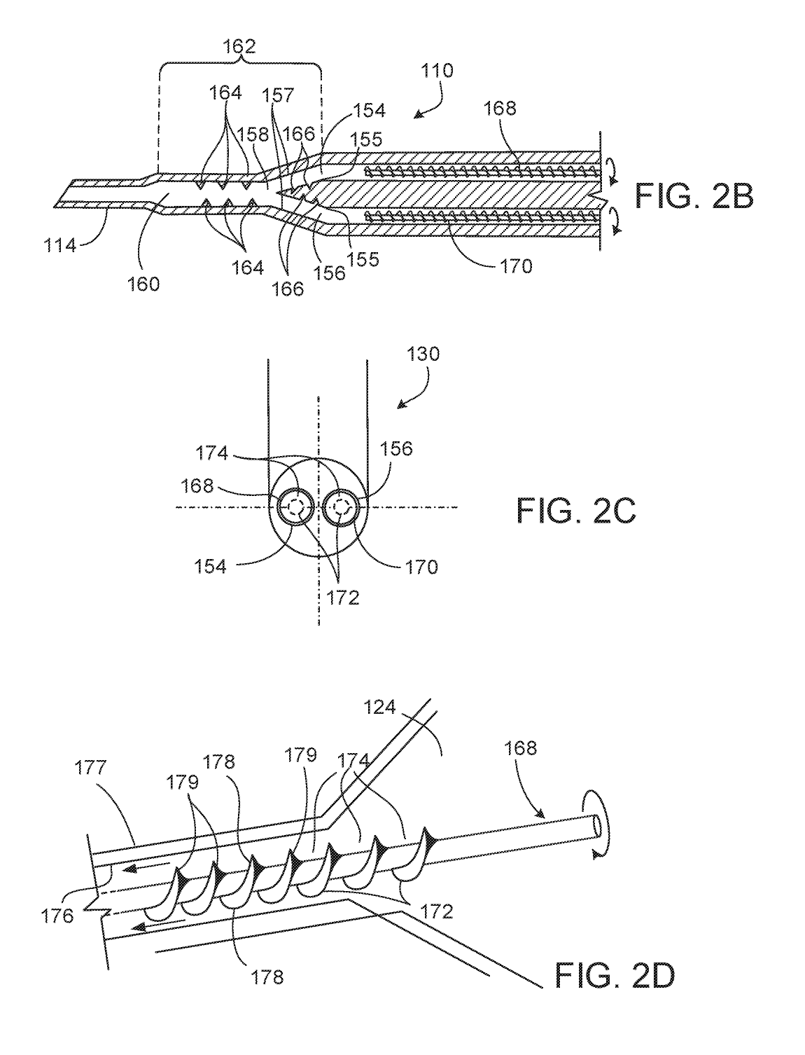

Referring to FIG. 1, an example system 100 provided herein can be used to deliver therapeutic agent deposits 140 to a left ventricle wall 184 of a heart 180 by advancing an injection catheter 102 through the aorta 182 and the aortic semilunar valve 183. The catheter system 100 shown in FIG. 1 includes the injection catheter 102 (which will be described in greater detail with FIGS. 2A-2D) having a distal portion 110, a proximal portion 120, and an elongate shaft 130 (e.g., a tubular body) therebetween. Distal portion 110 includes a distal tip, such as a needle 112, configured for penetrating tissue. The proximal portion 120 includes a manifold 122 coupleable to two therapeutic agent supplies 124,125 and a drive mechanism 126 to advance the therapeutic agents 124, 125 through the elongate shaft 130 and to dispense therapeutic agents 124, 125 from the needle 112 into the wall 184. As shown in the depicted embodiment, the drive mechanism 126 includes two motor drives 132, 134 and two sensors 136, 138. Each sensor 136, 138 can be coupled to a therapeutic agent supply source 124, 125 (e.g., a tank or a syringe) and electronically connected to a processor 142 to provide the system 100 with a feedback control loop for monitoring and regulating the motors 136, 138 and the flow rate of the dispensed therapeutic agents. In some cases, fluid delivery of the therapeutic agent can be controlled using a controller (not shown) and sensors 136, 138, to monitor the weight of the supply sources 124, 125, and to provide a controlled feedback system to adjust the rotational speed of the motors. In some cases, the drive mechanism 126 can include one or multiple (e.g., three or more) motor drives 132, 134 and/or sensors 136, 138. The system 100 can include first and second therapeutic agent supply sources 124, 125. In some cases, the volume of the therapeutic agent delivered into the heart can be controlled such that, for example, a ratio of two agent components (e.g., a first agent component and a second agent component) are dispensed at a 1:1 ratio.

The depicted elongate shaft 130 of FIG. 1 can include at least two lumens 154, 156 (shown in FIG. 2C) extending from the proximal portion 120 to the distal portion 110 of the catheter 102. Each lumen 154, 156 of the elongate shaft 130 can deliver a therapeutic agent, in either gel or liquid form, from the manifold 122, which may be supplied by one or multiple therapeutic agent supply sources 124, 125, to the distal portion 110 of the catheter 102. In some cases, the system 100 can deliver a first agent and a second agent each containing shear stress-sensitive therapeutic agents (e.g., cells) from the manifold 122 (e.g., the proximal end) to the needle 112 of the catheter. The catheter system 100 can optionally include a mixing region 162 (which will be discussed in greater detail with FIG. 2B) within the distal portion 110 of the catheter 102 for mixing the first agent with another agent, e.g., the second agent.

During use, the injection catheter system 100 can be introduced into a patient's left ventricle 186 (or other areas such as the atriums or the right ventricle) by advancing the catheter 102 through a lumen of a guide catheter (not shown). A guide catheter can be introduced and positioned within the patient's vasculature using conventional interventional techniques, for example, as described in U.S. Pat. No. 6,530,914 titled “Deflectable tip guide in guide system.” In some cases, the injection catheter 102 can be advanced within an area of the heart 180, e.g., the left ventricle 186, such that the needle 112 at the distal portion 110 of the system 100 can penetrate a target location along the heart wall 184. Advancement and withdrawal of the catheter 102 within the heart 180 can typically be achieved by a practitioner exerting axial force (e.g., pulling or pushing) on the proximal portion 120 of the injection catheter 102. Once the needle 112 has penetrated a desired location within the heart wall 184, a practitioner can actuate the drive mechanism 126 to advance the therapeutic agent through the catheter system 100 such that the agent injects from the needle 112 into the heart tissue 184, e.g., myocardium. The injection catheter 102 can be retracted from the injection site and advanced to penetrate another location along the heart wall 184 to inject the agents (e.g., therapeutic gels) into multiple sites within the myocardium 184.

Referring to FIGS. 2A-2D, the injection catheter system 100 of FIG. 1 includes a flexible, elongate shaft 130 adapted for providing the injection catheter 102 with flexibility for winding through a tortuous blood vessel pathway and gaining access to an interior region of a heart (e.g., the heart 180 of FIG. 1) for delivering the therapeutic agents. The depicted injection catheter system 100 includes an inner member 178 (which will be discussed in greater detail below with FIG. 2D) for delivering (or pumping) viscous therapeutic agents to the needle 112 (which may also be referred to as an injection port). The system, as shown, at least a portion of the shaft 130 includes two, separate lumens 154, 156 to deliver at least two different agent components to prevent the agent components from interacting prematurely (e.g., crosslinking) with one another prior to an injection.

In some cases, the injection catheter can include a drive mechanism (e.g., drive mechanism 126), an elongate, flexible tubular body (e.g., shaft 130), and one inner member (e.g., inner member 178). The shaft 130 can include a distal end portion, a proximal end portion, and defines a one lumen (e.g., lumen 154) extending therebetween. The inner member 178 can include a proximal end coupled to the drive mechanism and be rotatably disposed within the first lumen 154.

The injection catheter 102 and components thereof can include any suitable polymeric or metallic material. For example, in some cases, at least portions of the injection catheter 102, such as the elongate shaft 130, can be made from polymeric materials such as, but not limited to, polytetrafluoroethylene (PTFE), fluorinated ethylene propylene (FEP), Hytrel®, nylon, Picoflex®, Pebax®, and the like. In some cases, at least portions of the injection catheter 102, such as the needle 112, can be made from metallic materials such as, but not limited to, nitinol, stainless steel, stainless steel alloys (e.g., platinum-enriched stainless steel), titanium, titanium alloys, cobalt-based alloys (e.g., Elgiloy®), cobalt chrome, and combinations thereof. Elgiloy® is an alloy composition that includes cobalt, chromium, nickel, molybdenum and iron.

Referring to FIG. 2B, the distal portion 110 of the injection catheter 102 includes a tissue-penetrating needle. Various suitable types of needles 112 can be used for penetrating tissue, for example, the needle 112 can be a single bevel, a bi-bevel, a tri-bevel, or a trocar needle. Suitable sizes of the needle can range from about a 28-gauge needle to about a 22-gauge needle, or from about a 22-gauge to about a 15-gauge needle, including all values and ranges therebetween.

The distal portion 110 can include a first lumen 154 and a second lumen 156 that intersect at a junction 158 and merge into a single luminal passageway 160. The single lumen (e.g., a distal lumen) can be located distal to the junction 158. The junction 158 can, in some cases, be located at other locations of the catheter 102, for example, along a mid-portion or the proximal portion 120 of the system 100 or at the distal tip (e.g., the needle 112). In some cases, the injection catheter system 100 does not include a junction 158 for joining the two lumens 154, 156 of the shaft 130 such that two different therapeutic agents do not interact until the agents are injected into cardiac tissue (e.g., myocardium).

Still referring to FIG. 2B, the distal portion 110 can define an internal mixing chamber 162 located proximal to the distal tip (e.g., the needle 112) such that therapeutic agent components (not shown) can be mixed together within the system 100 before an injection. The internal mixing chamber may define a curvilinear fluid pathway adapted for mixing at least two therapeutic agents prior to dispensing the mixed agents from the needle. The internal mixing chamber 162 can include various channel features 164, 166 adapted to improve the mixing of two of more therapeutic agent components within the distal portion 110 of the system 100. As shown, the internal mixing chamber 162 can include internal features 166 that are defined along inner walls 155 of the first and second lumens 154, 156 that join the lumens 154, 156 at the junction 158. In some cases, the internal features can be defined along outer walls 157 of the first and second lumens 154, 156.

The internal features can include angled groves adapted to cause a rotational movement of a therapeutic agent passing though the lumen 154, 156. In some cases, the internal features 166 form a helical groove. In some cases, fluid can rotate in one direction on the larger diameter surface and another direction close to the internal features 166 creating a mixing zone in between opposite helical grooves. In some cases, a helical groove can switch direction at one point causing extra turning and mixing of the fluid.

The internal mixing chamber 162 can include a series of teeth 164 that create turbulence and mixing of the two therapeutic agent components. The agent mixture can then be injected though an injection needle 112. High pressure in the small diameter to low pressure in the large diameter of the distal portion of the catheter creates turbulence and therefore mixing due to an interruption of laminar flow, which keeps layers relatively static.

Referring to FIG. 2C, the elongate shaft 130 includes the first and second lumens 154, 156 for receiving first and second inner members 168, 170, respectively, that, in combination, can deliver two different therapeutic agents. More specifically, each inner member 168, 170 can have threads 172 (which will be discussed in greater detail with FIG. 2D) that provide one or more annular gaps 174 between the inner member 168, 170 and an inner wall 176 of the shaft 130 for receiving the therapeutic agent. In some cases, the annular gap can be a helically-shaped channel.

Referring back to FIG. 2A, the proximal portion 120 of the elongate shaft 130 can include a manifold 122 connecting two tubing lines 177, in which each tubing line has a fluid connection with the therapeutic agent supply sources such that two fluid pathways are maintained separate from each other but housed within a single tube member, such as the elongate shaft 130. For example, the manifold 122 can connect a first fluid pathway to a first therapeutic agent supply source to first lumen 154 of the elongate shaft 130 and connect a second fluid pathway to a second lumen 156 of the elongate shaft 130. Combining the tubing lines 177 to a single tube member, such as the elongate shaft 130, can provide a practitioner with increased ease of handling during the advancement and retraction of the system 100 during use.

Referring the FIG. 2D, a proximal region of the system 100 can include a tapered transitional region between each supply source 124, 125 and each tubing line 177. As shown, the first inner member 168 can extend from a luminal region of the supply 124 to one of the tubing lines 177 and along at least a portion of the elongate shaft 130. In some cases, the first inner member 168 can extend longitudinally along the entire length of the elongate shaft 130.

The depicted embodiment of the inner member 168 has a helical feature 178 (which can also be described as an auger-shaped member, or a threaded feature). The helical feature 178 can form a continuous, interstitial cavity (or channel) for receiving a therapeutic agent (e.g., a therapeutic gel) and transporting the agent distally along the catheter device 102 or catheter system 100 as the inner member 168 is rotated within the lumen of the elongate shaft 130 and the tubing lines 177. In some cases, a fluid pathway is formed by each interstitial cavity, in which each fluid pathway is defined a minimum diameter of each interstitial cavity and the lumen walls of the tubular body. Each interstitial cavity is adapted to receive a therapeutic agent, which can have a viscosity ranging from about 0.1 pascal-seconds (100 cP) to about 100 pascal-seconds (100,000 cP). In some cases, the injection catheter 102 is adapted for delivering a first therapeutic agent within a first continuous pathway defined by the interstitial cavity of a first lumen and the first inner member 168 and a second therapeutic agent in a second continuous pathway defined by the interstitial cavity of a second lumen and the second inner member 170. The inner members 168, 170 of the system 100 can include the helical feature 178 with a beveled edge 179. In some cases, the beveled edge 179 can increase the volume within the catheter system for containing the therapeutic agent.

The helical feature 178 of the inner member 168 can be dimensioned as desired. For example, in some cases, the helical feature 178 of each inner member 168 can be formed by threads having an amplitude ranging from about 0.1 millimeters to about 0.9 millimeters, a pitch ranging from about 0.1 millimeters to about 2.0 millimeters, and a thread groove width ranging from about to about 0.1 millimeters to about 3 millimeters. In some cases, the helical feature 178 of the inner member 168 can be defined by a ratio of a maximum diameter to a minimum diameter that ranges from about 0.2 to about 0.9. In some cases, the ratio of the groove width (GW) to pitch of the helical feature 178 can range from about 2 to about 20. In some cases, the ratio of the maximum outer diameter of the helical feature 178 to the inner diameter of the catheter lumen can range from about 0.1 to about 1.

In some cases, the devices and systems provided herein can contain one or more inner members 168, 170 that include cylindrical portions (e.g., portions having no threads) to allow each inner member 168, 170 to have increased flexibility or bending radius in desired catheter regions. For example, at least a portion of the inner member can include a cylindrical portion that allows the injection catheter to bend with greater flexibility at a patient's aortic arch. In some cases, an example inner member can have one or more helical features and one or more cylindrical portions. For example, in some cases, an inner member can include two, separate helical features in which the helical portions features are separated by a cylindrical portion.

Because the therapeutic agent (e.g., therapeutic gel) can be advanced through the length of the catheter 102 within the catheter lumen via a rotational mechanism, the therapeutic agent is able to move through a small lumen diameter without the use of excessive force and pressure. For systems and devices that include a hand-operated syringe, it can be advantageous to have the system or device configured for injecting a therapeutic agent with a force of less than 50 newtons (N) to minimize damage to the therapeutic agent. Also, forces greater than 50 N can make it difficult for a practitioner to operate the device, especially when using a hand-operated syringe. For systems and devices that include machine-operated injectors, greater force and pressure values may be achieved, but these forces and pressures (e.g., stresses greater than 300 PSI (35 MPa)) may damage the catheter components as well as the therapeutic agent. Advancing the therapeutic agent by rotationally translation therefore provides an important benefit of reducing the shear stresses that are imposed on the therapeutic agent during an injection such that the agent can be injected into tissue without being compromised or substantially compromised prior to delivery.

In some cases, as shown, the inner member 168, 170 can be rotated at a speed sufficient to propel the therapeutic agent (e.g., a viscous liquid) distally within the shaft 130 of the injection catheter 102. In some cases, the inner member 168, 170 of the devices and system 100 provided herein can rotate at a speed ranging from greater than 0 rotations per minute (RPM) to about 3000 RPM (e.g., from about 10 RPMs to about 500 RPM, from about 500 RPM to about 1000 RPM, from about 1000 RPM to about 2000 RPM, or from about 2000 RPM to about 3000 RPM). The inner member 168, 170 can rotate, in some cases, at a speed of greater than 3000 RPM.

In various cases, the systems 100 and devices 102 provided herein can include an inner member 168, 170 configured for effectively shearing thin viscous fluids along an entire length of a catheter 102 such that the viscous fluids can more easily flow through the catheter 102. Some viscous fluids have a shear thinning property, that is, as shear stress is applied to the fluid the viscosity reduces substantially. For example, in some cases, the viscosity of an exemplary viscous fluid, such as a hyaluronic acid solution, can reduce from about 50% to about 90%. In some cases, the viscosity of an exemplary viscous fluid (e.g., hyaluronic acid solution) can reduce from about 2 pascal-seconds to about 1 pascal-seconds (or from about 2000 cP to about 1000 cP). The shearing stress generated from shearing fluids using the inner member 168, 170 in the systems and devices provided herein can be significantly lower than the shearing stress that can damage cells (e.g., about 500 Pa). For example, in some cases, the systems and devices provided herein can generate a maximum shear stress of about 50 Pa. In some cases, the systems and devices provided herein can generate a shear stress of less than 50 Pa, or greater than 50 Pa (e.g., about 100 Pa, about 150 Pa, about 200 Pa, about 250 Pa, about 300 Pa, about 350 Pa, about 400 Pa, or about 450 Pa).

In some cases, the therapeutic agents delivered by the systems and devices provided herein can have a viscosity of greater than 1,000 cP. In some cases, the therapeutic agents can have a viscosity of greater than 5,000 cP. In some cases, the therapeutic agents can have a viscosity of 10,000 cP or more. In some cases, the therapeutic agent can include saline, cells, blood serum, or another physiologically relevant and/or compatible fluid. All viscosities discussed herein are viscosities at body temperature unless otherwise indicated. In some cases, viscosity can be determined using a standardized measurement protocol, such as ASTM D 2983.

In some cases, the therapeutic agents delivered using methods, device, systems, and kits provided herein can come as two or more components that are mixed at the site of injection. In some cases, the two or more agent components can cross-link at the site of the injection to form a gel. For example, some pre-gel therapeutics, which can be delivered using methods, systems, devices, and kits provided herein, can include two agent components that are intended to be mixed equally at the site of injection to crosslink to form a therapeutic gel. In some cases, one or more agent components can include cells (e.g., cardiopoietic stem cells). In some cases, a first agent component and a second agent component can be mixed in various suitable ratios that range from about 10:90 to about 50:50. The mixing ratio can optionally be controlled by the rotation of the augers by one or more feedback controllers. In some cases, one or both agent components can have a viscosity of 100 cP or greater. In some cases, one or both agent components can have a viscosity of 500 cP or greater, 1,000 cP or greater, 5,000 cP or greater, or 10,000 cP or greater. In some cases, both agent components can have the same viscosity. In some cases, the agent components can each have a viscosity less than the viscosity of a gel resulting from the mixture of the agent components. In some cases, a cross-linked gel resulting from two or more agent components being mixed can have a viscosity of greater than 10,000 cP, greater than 100,000 cP, or greater than 1,000,000 cP. In some cases, the cross-linked gel resulting from two or more agent components can be a solid.

Mixing different agent components to create a higher viscosity agent (e.g., gel) or solid can result in the clogging of passages in an injection device or system. In some cases, devices and systems provided herein can include a detachable tip including intersecting channels that mix two or more agent components. In some cases, methods provided herein include a step of separating a detachable tip from the remainder of a device provided herein between injections into anatomical locations to clean it out and replace it. In some cases, methods provided herein include a step of separating a detachable tip from a remainder of a device provided herein between injections into anatomical locations to replace it with a new detachable tip. In some cases, systems provided herein can include multiple detachable tips each having intersecting channels to mix two or more agent components for each injection device. In some cases, kits provided herein can include at least 2 detachable tips for each injection catheter. In some cases, kits provided herein can include at least 3, at least 5, at least 8, or at least 10 detachable tips for each injection catheter.

In some cases, the therapeutic agent can include a shearing thinning material (e.g., gel) such as hyaluronic acid based gels. Suitable exemplary hyaluronic acid based gels include, but are not limited to, a hylan-B gel (e.g, Hylaform®), a divinyl sulfone-crosslinked hyaluronic acid hydrogel, a methacrylated hyaluronic acid, an acrylated hyaluronic acid and a PEG-SH(4) crosslinker, a thiol-modified hyaluronic acid, and a tyrosine-derived hyaluronic acid, a polyethylene oxide gel. See Anna Gutowska et al., Injectable Gels for Tissue Engineering, The Anatomical Record, 263:4, 342-349 (2001). In some cases, the shearing thinning material can include xanthan gum solutions. In some cases, an exemplary solid gel can shear thin before forming a hydrogel solid, such as a β-Hairpin peptide-based hydrogel, which is a class of injectable hydrogel solids, which has significant future potential use in injectable therapies. In some cases, β-hairpin peptide hydrogels may be injected as preformed solids, because the solid gel can shear-thin and consequently flow under a proper shear stress but immediately recover back into a solid on removal of the stress. See Congqi Yan et al., Injectable solid hydrogel: mechanism of shear-thinning and immediate recovery of injectable β-hairpin peptide hydrogels, Soft Matter, 20:6, 5143-5156 (2010). In some cases, the therapeutic agent can contain one or more bioadhesive materials (e.g., bioadhesive hydrogels) that include, but are not limited to, fibrin, albumin, polyurethane, cyanoacrylates, albumin-glutaraldehyde, and combinations thereof.

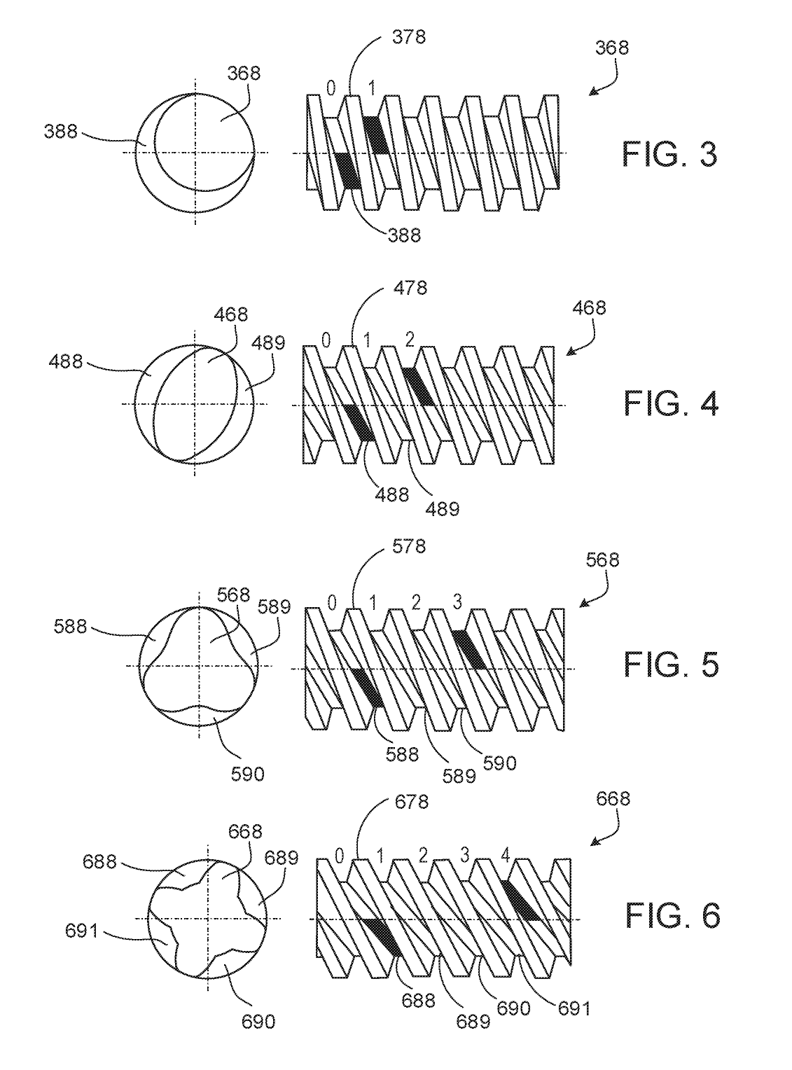

Referring to FIGS. 3-6, an inner member 368, 468, 568, 658 of the devices and systems provided herein can include one or more helical features 378, 478, 578, 678 (e.g., threads), which can also be described as one or more starts. Referring to FIG. 3, the inner member 368 has a single-start design, meaning that there is only one helical feature 378 wrapped around a cylinder portion of the inner member. As shown by this depicted embodiment, the inner member 378 is defined by one helical feature 378 (e.g., helix) that forms a gap 388 longitudinally extending as a continuous spiral-shaped channel, enumerated by 0 and 1 in FIG. 3. Referring to FIGS. 4-6, in some cases, the inner member 468, 568, 658 has multiple starts, in which at least two helical features 478, 578, 678 are wrapped around a cylinder portion of the inner member 468, 568, 658. Certain embodiments of the devices and systems provided herein can therefore contain an inner member 468, 568, 658 with multiple starts in which multiple helical features 478, 578, 678 form multiple channels. For example, in some cases, the inner member 468, 568, 658 can include two starts (enumerated by 0, 1, and 2 in FIG. 4), three starts (enumerated by 0, 1, 2, and 3 in FIG. 5), four starts (enumerated by 0, 1, 2, 3, and 4 in FIG. 6), or more than four starts. The inner member 468 with a two-start design can form a first gap 488 and a second gap 489. The inner member 568 with a three-start design can form a first gap 588, a second gap 589, and a third gap 590. The inner member 668 with a four-start design can form a first gap 688, a second gap 689, a third gap 690, and a fourth gap 691. Increasing the number of helices (e.g., channels) can provide a device or system with the benefit of increasing the volume of the therapeutic agent delivered per every rotation of the inner member.

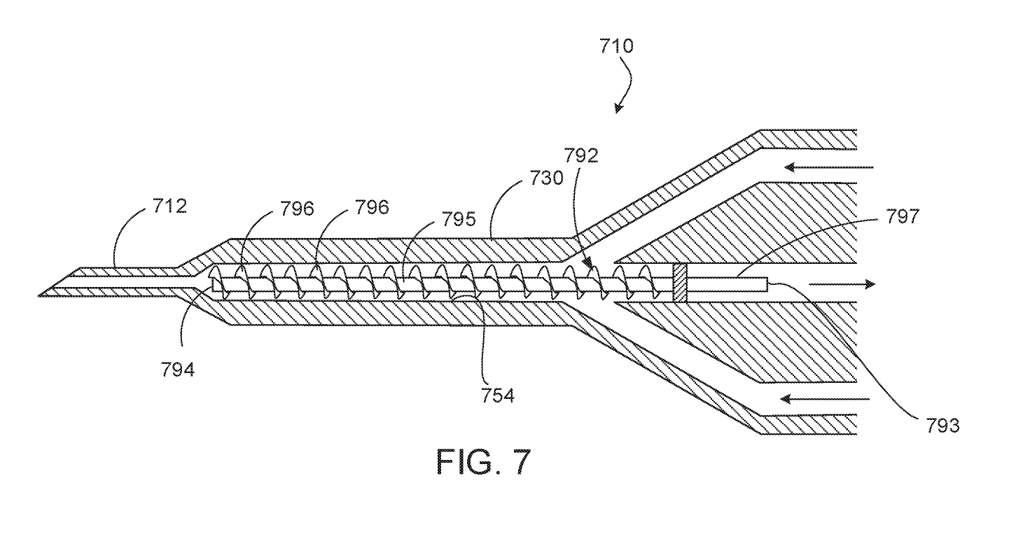

Referring to FIG. 7, another embodiment of the devices and systems provided herein has a distal portion 710 containing a supplemental inner member 792, which includes a proximal end 793 and a distal end 794 with a distal helical element 795. The depicted distal helical element 795 includes threads 796 disposed about a rod portion 797 of the supplemental inner member 792 at its distal portion. As shown in the depicted embodiment, the distal helical element 795 is disposed within the lumen 754 of the injection catheter device just proximal to a needle 712. In some cases, the distal helical element 795 is disposed about at least a portion of the cylindrical rod portion 797. For example, as shown in FIG. 7, the distal helical element 795 is disposed about a rod portion at the distal portion of the supplemental inner member 792 such that the remaining portions of the supplemental inner member 792 can extend through a catheter shaft 730 with the more flexible smaller diameter rod. In some cases, the entire length or a substantial portion of the supplemental inner member 792 can include a threads 796 disposed about the rod portion 797. The distal helical element 795 can be configured for advancing therapeutic agents through the distal portion of a catheter. The distal helical element 795 can optionally be configured for mixing two or more pre-gel components such that the agent can be sufficiently agitated prior to being dispensed from the needle 712.

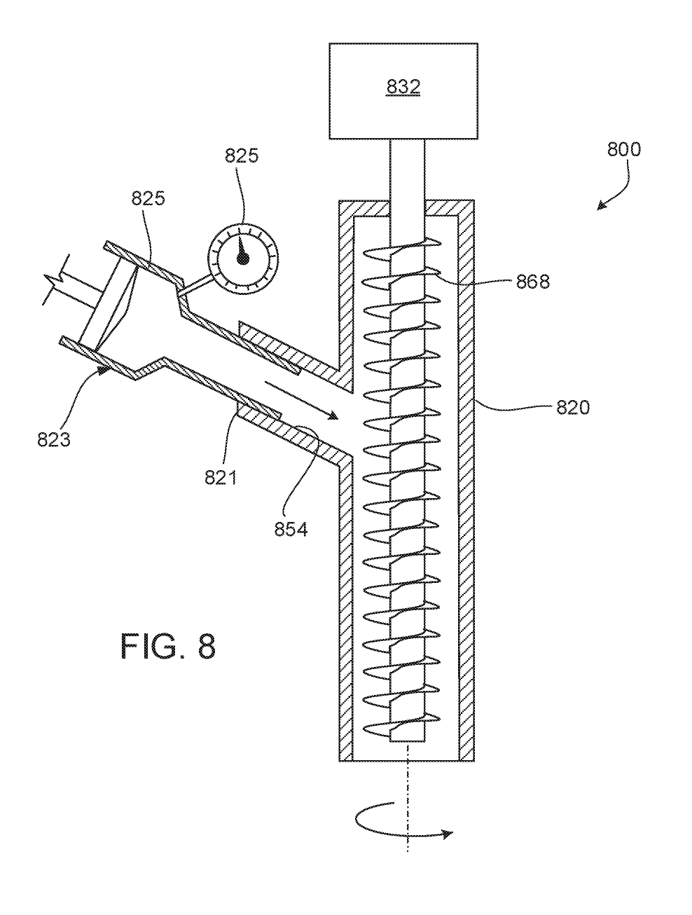

Referring to FIG. 8, another example device 800 provided herein can include an inlet 821 adapted for coupling to a pressure-generating assembly 823 configured for providing a pressurized supply of a viscous fluid (e.g., therapeutic agent). In some cases, the pressure-generating assembly 823 includes a human-actuated syringe 825, or a machine-actuated supply (not shown). The pressure-generating assembly 823 can optionally include a pressure gauge 827 for providing pressure measurements to a user during use. As shown by the depicted embodiment of FIG. 8, the inlet 821 for the pressure-generating assembly 823 can be a side hole located along a side wall of catheter at its proximal portion 820. The lumen 854 of the inlet 821 can provide a fluid pathway that is defined by a gap between the inner member 868 and the surrounding lumen 854 of the injection catheter. The inner member 868 may be actuated by a motor 832. In some cases, the pressure-generating assembly 823 can apply a pressure less than or equal to 207 MPa (30 pounds per square inch) to the viscous fluid within the injection catheter. The pressure-generating assembly 823 can provide the benefit of supplying the pressurized viscous fluids such that the fluids can be transported through the catheter lumen 854 using a reduced revolutions per minute (RPM) value and under lower shear stresses, as compared to transporting non-pressurized viscous fluids through the catheter lumen 854.

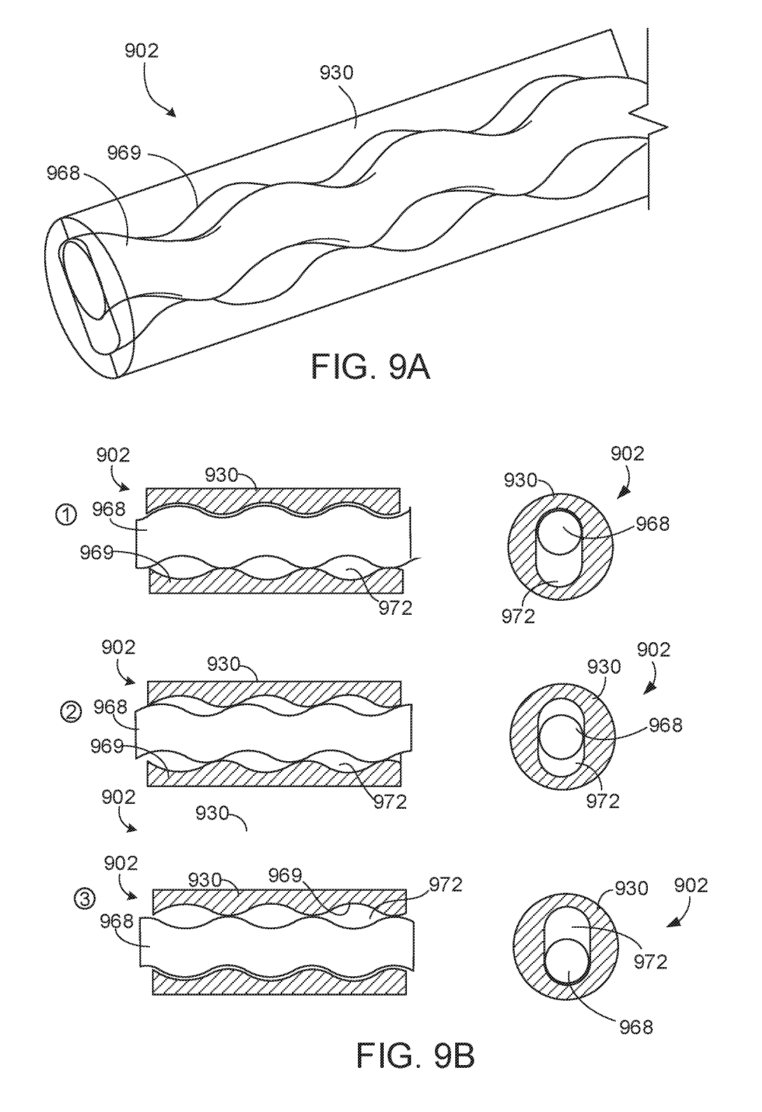

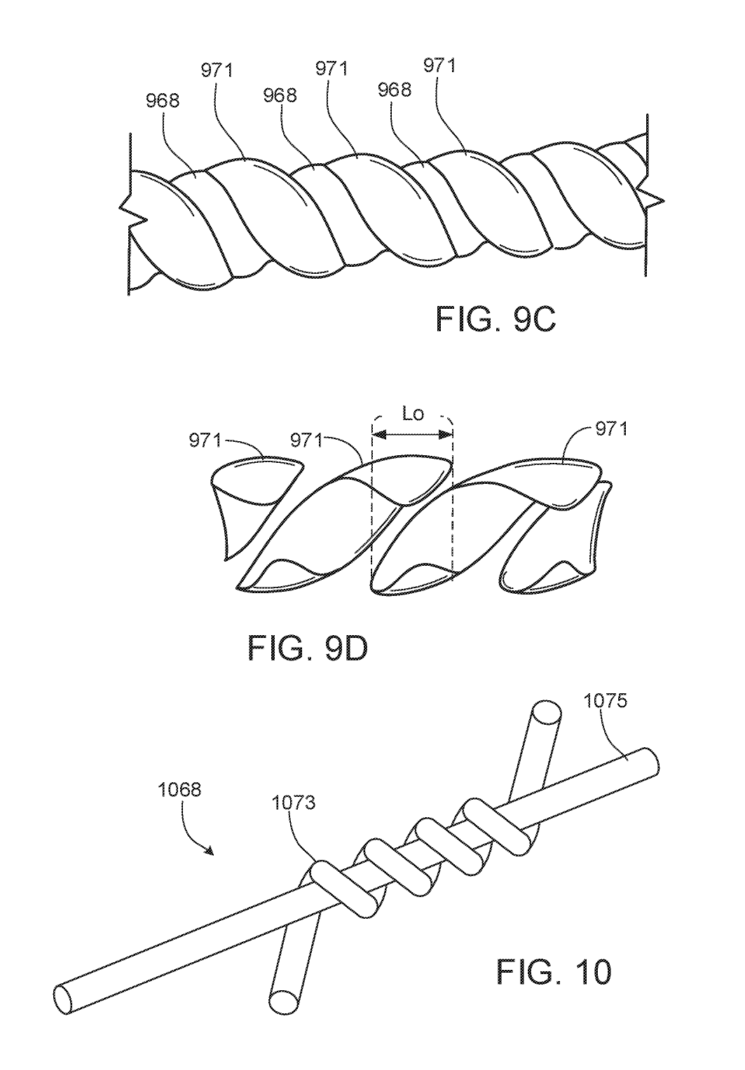

Referring to FIGS. 9A-9D, an alternative embodiment of an injection catheter system 900 includes a progressive cavity design (also referred to as an eccentric screw or a worm drive design) in which a helically-shaped inner member 968 with a circular cross-section rotates within an elongate lumen 969 of an elongate shaft 930. The elongate lumen, in a transverse cross section, can have an oblong outline defined by a pair of spaced concave semi-circular ends and sides joining the semi-circular ends. In some cases, the elongate lumen 969 can extend longitudinally along the shaft 930 in a double helix pattern. The helically-shaped inner member 968 may be eccentrically driven within the elongate lumen 969. In particular, the inner member 968 can rotate within the elongate lumen 969, as shown in FIG. 9B, to transport discrete volumes of a therapeutic agent (e.g., a therapeutic gel), as shown in FIG. 9C. The inner member 968 and luminal walls of the elongate shaft 930 can together form pockets, or a discrete cavities, having a fixed shape. Best shown in FIG. 9D, the fixed volumes of a therapeutic agent can be delivered in the discrete cavities 972 formed between the inner member 968 and the lumen 969 of the elongate shaft 930. More specifically, as the inner member 968 is rotated within the lumen 969 of the elongate shaft 930, the therapeutic agent 971 within the discrete cavities 972 can progress distally along the lumen 969 of the injection catheter system 900, resulting in the injection catheter system 900 having a volumetric flow rate proportional to the rotation rate while applying low levels of shear stress to the delivered therapeutic agent. The progressive cavity design can provide a non-pulsating, continuous output since each discrete cavity overlaps a predetermined longitudinal length (depicted by Loin FIG. 9D) in the longitudinal direction with an adjacent discrete cavity. In some cases, the progressive cavity design includes a pump that can be operated from greater than 0 RPM to about 500 RPM, including all values and ranges therebetween.

The progressive cavity design can be configured to deliver therapeutic agents (e.g., gels) of varying viscosities. In some cases, the progressive cavity design can be highly advantageous because the pump design can deliver highly viscous liquids, such as liquids that include semi-solid slurries and/or particles. Accordingly, certain embodiments of the devices and system provided herein can be well-suited for delivering liquids having a viscosity ranging from about 0.1 pascal-seconds (100 cP) to about 100 pascals-seconds (100,000 cP), or shear-sensitive materials. Other examples of therapeutic agents that can be delivered by the device and system embodiments provided herein, include, but are not limited to, therapeutic pastes, adhesives, and crystalline drug slurries.

### Methods of Manufacturing an Injection Catheter

A method of manufacturing an injection catheter can include the use of a combination of various processes known to those skilled in the art. For example, suitable processes for manufacturing an injection catheter can include, but are not limited to, injection molding, extrusion, laser welding, adhesive bonding, metal drawing, soldering and combinations thereof.

In some case, a tubular body, and other tubular components, of an injection catheter of the systems and devices provided herein can be made from various processes for fabricating metallic tubes and/or plastic tubes. Suitable processes, in some cases, can include, but are not limited to, extrusion and drawing processes. In some cases, the inner member of an injection catheter can also be made from processes for making solid elongate members (e.g., rods) such as extrusion and drawing. The inner member can optionally include additional processing, such as a winding process, a laser etching process, a laser cutting process, an adhesive bonding process, a soldering process, a laser welding process, and combinations thereof. In some cases, metal and/or polymer components of the injection catheter can be made using an additive manufacturing technology. Additive manufacturing technology can be advantageous for achieving components that intricate curvilinear designs interior cavities, such as components that include a mixing zone, or a mixing chamber, as discussed herein.

Referring to FIG. 10, a method of manufacturing an inner member 1068 of an injection catheter can include obtaining a first elongate member 1073 and a second elongate member 1075 and deformably winding the first elongate member 1073 about the second elongate member 1075, such that the first elongate member 1073 forms a helical feature around the second elongate member 1075. In some cases, the first and second elongate members 1073, 1075 may be bonded together using at least one of adhesive bonding, soldering, and welding. For example, the inner member 1068 with a helical feature can be constructed by wrapping a wire (e.g., the first elongate member 1073) around a central rod (e.g., the second elongate member 1075) and welding the wire at various points to the central rod to affix the wire in place. In some cases, the first elongate member 1073 can be wound about the second elongate member 1075 such that the first elongate member 1073 applies a compressional force on the second member 1075, thus creating an interference fit between the first and second elongate members. In some cases, the first elongate member 1073 may be wound about the second elongate member 1075 with a constant pitch, or a variable pitch. The first elongate member 1073 can have a round, a square, an oval, or a rectangular cross-sectional shape. In some cases, the first elongate member 1073 into a desired final form by using, for example, a laser cutting process to shape the elongate member before or after the winding process.

In some cases, multiple elongate members can be wound about a second elongate member. For example, a first elongate member and a third elongate member can be deformably wound about a second elongate member, in some cases. In some cases, the first and third elongate member can have different cross-sectional shapes and/or dimensions.

Further details on alternative systems configured for delivering a therapeutic agent (e.g., gel) into heart tissue are disclosed in Appendix A and herein incorporated by reference in its entirety. It should be understood that one or more design features of the devices and systems provided herein can be combined with other features of other devices provided herein. In effect, hybrid designs that combine various features from two or more of the device and system designs provided herein can be created, and are within the scope of this disclosure.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in an embodiment. Conversely, various features that are described in the context of an embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

In addition to being directed to the teachings described above and claimed below, devices and/or methods having different combinations of the features described above and claimed below are contemplated. As such, the description is also directed to other devices and/or methods having any other possible combination of the dependent features claimed below.

Numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein. All references, publications, and patents referred to herein, including the figures and drawings included therewith, are incorporated by reference in their entirety.

What is claimed is:

1. An injection catheter comprising:
a drive mechanism;
an elongate, flexible tubular body having a distal end portion, a proximal end portion, and defines a first lumen extending therebetween; and
a first inner member including a proximal end coupled to the drive mechanism and rotatably disposed within the first lumen;
wherein the first inner member and the first lumen defining an annular gap therebetween configured to deliver a therapeutic agent contained therein from the proximal end portion to the distal end portion of the tubular body when the first inner member is rotated within the lumen such that shear stresses exerted on the therapeutic agent are less than 500 pascals.

2. The injection catheter of claim 1, wherein first inner member includes a helical feature formed along at least a portion of the first inner member and the annular gap is partially defined by an interstitial channel of the helical feature.

3. The injection catheter of claim 1, further comprising a needle coupled to the distal end of the tubular body and a manifold coupled to the proximal end of the tubular body, wherein the manifold is connectable to multiple therapeutic agent supply sources.

4. The injection catheter of claim 1, wherein the tubular body further defines a second lumen extending from the proximal end portion to the distal end portion and a second inner member that has a proximal end coupled to the drive mechanism, wherein the second inner member is rotatably disposed within the second lumen.

5. The injection catheter of claim 3, further comprising a distal portion between the needle and the distal end of the tubular body, the distal portion comprising an inner junction that joins the first and second lumens of the tubular body together into a distal lumen.

6. The injection catheter of claim 5, wherein the distal lumen is located distal to the inner junction and defines a curvilinear fluid pathway adapted for mixing at least two therapeutic agents within the distal lumen prior to dispensing the mixed agents from the needle.

7. The injection catheter of claim 2, wherein the interstitial channel is configured to receive a therapeutic agent having a viscosity ranging from about 100 centipoises to about 100,000 centipoises.

8. The injection catheter of claim 2, wherein the helical feature of each inner member is formed by threads having an amplitude ranging from about 0.1 millimeters to about 0.9 millimeters, a pitch ranging from about 0.1 millimeters to about 2.0 millimeters, and a thread groove width ranging from about to about 0.1 millimeters to about 3 millimeters.

9. The injection catheter of claim 3, wherein the needle has a needle size ranging from about 28 gauge to about 15 gauge.

10. The injection catheter of claim 1, wherein the inner member comprises nitinol, stainless steel, stainless steel alloys, cobalt-based alloys, titanium, titanium alloys, or combinations thereof.

11. The injection catheter of claim 1, further comprising two sensors and a feedback controller, wherein each sensor is coupled to a therapeutic agent supply source to monitor volume of a therapeutic agent within the supply source.

12. The injection catheter of claim 1, further comprising at least two therapeutic agent components that are intended to be mixed equally at the site of injection to crosslink to form a therapeutic gel.

13. An injection catheter comprising:
an elongate, flexible tubular body having a distal end portion, a proximal end portion, and at least one lumen extending therebetween; and
a means for delivering a therapeutic agent within the at least one lumen from the proximal end portion to the distal end portion of the tubular body such that shear stresses exerted on the therapeutic agent are less than 500 pascals (Pa).

14. A method of manufacturing an injection catheter, the method comprising deformably winding a first elongate member about a second elongate member such that the first elongate member forms a helical feature around the second elongate member.

15. The method of claim 14, wherein the first and second elongate members are bonded together using at least one of adhesive bonding, soldering, and welding.

16. The method of claim 14, wherein the first elongate member is wound about the second elongate member such that the first elongate member applies a compressional force on the second member such that an interference fit is produced between the first and second elongate members.

17. The method of claim 14, wherein the first elongate member is wound about the second elongate member with a variable pitch.

18. The method of claim 14, wherein the first elongate member has a round, a square, an oval, or a rectangular cross-sectional shape.

19. The method of claim 14, further comprising a laser cutting process to shape the first elongate member into a final form.

20. The method of claim 14, further comprising obtaining a third elongate member and deformably winding the third elongate member about the second elongate member.
